(19) United States
(12) Patent Application Publication
(10) Pub. No.: US 2023/0220296 A1
(43) Pub. Date: Jul. 13, 2023

(54) METHOD FOR IMPROVING OIL YIELD FROM GERM IN A WET MILLING PROCESS

(71) Applicant: Novozymes A/S, Bagsvaerd (DK)

(72) Inventor: Thomas Patrick Gibbons, Wake Forest, NC (US)

(73) Assignee: Novozymes A/S, Bagsvaerd (DK)

(21) Appl. No.: 18/010,643

(22) PCT Filed: Jul. 1, 2021
(86) PCT No.: PCT/US2021/040056

§ 371 Date: Dec. 15, 2022

Related U.S. Application Data
(60) Provisional application No. 63/048,076, filed on Jul. 3, 2020.

(51) Int. Cl. C11B 1/02; C12N 9/24; C12N 9/42

(52) U.S. Cl. CPC C11B 1/025; C12N 9/248; C12N 9/2437; C12Y 302/01004

(57) ABSTRACT

The present invention provides a method for improving oil yield from germ in a wet milling process, the method comprising admixing a process stream comprising corn germ with an enzyme composition comprising an effective amount of one or more hydrolytic enzymes, wherein at least one of said hydrolytic enzymes is a xylanase polypeptide selected from the group consisting of: GH5, GH10, GH30, GH11 polypeptides.

### FIELD OF THE INVENTION

The present invention relates to a method for improving the total oil yield from germ in a wet milling process.

### BACKGROUND OF THE INVENTION

Conventional wet milling of corn is a process designed for the recovery and purification of starch and several coproducts including germ, gluten, starch, fiber, and oil.

Fiber is the least valuable coproduct, so the industry has put substantial effort into increasing the yield of the more valuable products, such as starch and gluten, while decreasing the fiber fraction. High quality starch is valuable as it can be used for a variety of commercial purposes after further processing to products such as dried starch, modified starch, dextrins, sweeteners and alcohol. Gluten is usually used for animal feed, as corn gluten meal (Around 60% protein) or corn gluten feed (Around 20% protein).

The wet milling process can vary significantly dependent on the specific mill equipment used, but usually the process include: grain cleaning, steeping, grinding, germ separation, a second grinding, fiber separation, gluten separation and starch separation. After cleaning the corn kernels, they are typically softened by soaking in water or in a dilute SO₂ solution under controlled conditions of time and temperature. Then, the kernels are grinded to break down the pericarp and the germ is separated from the rest of the kernel. The remaining slurry, mainly consisting of fiber, starch and gluten is finely ground and screened in a fiber washing process, to separate the fiber from starch and gluten, before the gluten and starch is separated and the starch can be purified in a washing/filtration process.

The use of enzymes in several steps of the wet milling process has been suggested, such as the use of enzymes for the steeping step of wet milling processes. The commercial enzyme product Steepzyme® (available from Novozymes A/S) has been shown suitable for the first step in wet milling processes, i.e., the steeping step where corn kernels are soaked in water.

More recently, “enzymatic milling”, a modified wet milling process that uses proteases to significantly reduce the total processing time during corn wet milling and eliminates the need for sulfur dioxide as a processing agent, has been developed. Johnston et al., Cereal Chem, 81, p. 626-632 (2004).

U.S. Pat. No. 6,566,125 discloses a method for obtaining starch from maize involving soaking maize kernels in water to produce soaked maize kernels, grinding the soaked maize kernels to produce a ground maize slurry, and incubating the ground maize slurry with enzyme (e.g., protease).

U.S. Pat. No. 5,066,218 discloses a method of milling grain, especially corn, comprising cleaning the grain, steeping the grain in water to soften it, and then milling the grain with a cellulase enzyme.

WO 2002/000731 discloses a process of treating crop kernels, comprising soaking the kernels in water for 1-12 hours, wet milling the soaked kernels and treating the kernels with one or more enzymes including an acidic protease.

WO 2002/000911 discloses a process of starch gluten separation, comprising subjecting milled starch to an acidic protease.

WO 2002/002644 discloses a process of washing a starch slurry obtained from the starch gluten separation step of a milling process, comprising washing the starch slurry with an aqueous solution comprising an effective amount of acidic protease.

WO 2014/082566 and WO 2014/082564 disclose cellulolytic compositions for use in wet milling.

Methods for increasing oil extraction from starch containing material has previously been disclose, e.g., in WO92/20777 which discloses adding an acid stable fungal protease during fermentation in a starch to ethanol process.

While the art has investigated the effect of using enzymes in corn wet milling, during steeping/soaking of corn kernels, during grinding of the corn kernels and in starch gluten separation, there is still a need for improved enzyme technology that may lower the energy expenditure and costs associated with corn wet milling and provide increased yield of oil.

### BRIEF DESCRIPTION OF THE FIGURES

FIG. 1: Schematic of enzyme treatment of germ, composition analysis and composition mass determination

### SUMMARY OF THE INVENTION

The present invention relates to a method for improving oil yield from germ in a wet milling process, the method comprising admixing a process stream comprising corn germ with an enzyme composition comprising an effective amount of one or more hydrolytic enzymes, wherein at least one of said hydrolytic enzymes is a xylanase polypeptide selected from the group consisting of: GH5, GH10, GH30, GH11 polypeptides.

Definition of Enzymes:

Arabinofuranosidases/polypeptide with arabinofuranosidase activity: The term “arabinofuranosidase” means an alpha L-arabinofuranoside arabinofuranohydrolase (EC 3.2.1.55) that catalyzes the hydrolysis of terminal non-reducing alpha-L-arabinofuranoside residues in alpha-L-arabinosides. The enzyme acts on alpha-L-arabinofuranosides, alpha-L-arabinans containing (1,3)- and/or (1,2)- and/or (1,5)-linkages, arabinoxylans, and arabinogalactans. Alpha-L arabinofuranosidase is also known as arabinosidase, alpha-arabinosidase, alpha-L-arabinosidase, alphaarabinofuranosidase, polysaccharide alpha-L-arabinofuranosidase, alpha-L-arabinofuranoside hydrolase, L-arabinosidase, or alpha-L-arabinanase. Arabinofuranosidase activity can be determined using 5 mg of medium viscosity wheat arabinoxylan (Megazyme International Ireland, Ltd., Bray, Co. Wicklow, Ireland) per ml of 100 mM sodium acetate pH 5 in a total volume of 200 μl for 30 minutes at 40° C. followed by arabinose analysis by AMINEX® HPX-87H column chromatography (Bio-Rad Laboratories, Inc., Hercules, Calif., USA). Arabinofuranosidases can be found in, e.g., the GH43, GH62, GH51 families.

Beta-glucanase/polypeptide with beta-glucanase activity: The term “beta-glucanase” encompasses polypeptides having beta-1,6-glucanase activity and/or exo- and/or -endo beta-1,3-glucanase activity. As used herein, “polypeptide having beta-1,6-glucanase activity and/or exo- and/or -endo beta-1,3-glucanase activity” means that the polypeptide exhibits at least one of these activities, but may also possess any combination of these activities, including all the activities. The term “exo- and/or -endo beta-1,3-glucanase” encompasses polypeptides that have either exo- and/or -endo beta-1,3-glucanase activity, both exo- and/or -endo beta-1,3-glucanase activities, as well as polypeptides having mixed beta-1,3(4) and/or beta 1,4(3)-glucanase activities. Preferably, the polypeptides having beta-1,6-glucanase activity and/or exo- and/or -endo beta-1,3-glucanase activity are members of a glycoside hydrolase family selected from GH30, for instance GH30_3, GH5, for instance GH5_15, GH16, GH55, for instance GH55_3, GH64, and GH131, for instance GH131A and GH131B.

In one aspect, “beta-glucanase” means polypeptides having beta-1,6-glucanase activity referred to as 6-β-D-glucan glucanohydrolase (EC 3.2.1.75) that catalyze the random hydrolysis of (1→6)-linkages in (1→6)-β-D-glucans. In addition to acting on 1,6-oligo-beta-D-glucosides, members of this family of enzymes also act on lutean and pustulan. These beta-glucanases include members of the GH30_3, GH5_15 and GH131A and GH131B families. For purposes of the present invention, beta-1,6-glucanase activity is determined according to the procedure described in the Examples. In one aspect, the beta-glucanase polypeptides have at least 20%, e.g., at least 40%, at least 50%, at least 60%, at least 70%, at least 80%, at least 90%, at least 95%, or at least 100% of the beta-1,6-glucanase activity of the mature polypeptide of SEQ ID NO: 7 Beta-glucosidase/polypeptide with beta-glucosidase activity: The term “beta-glucosidase” means a beta-D-glucoside glucohydrolase (E.C. 3.2.1.21) that catalyzes the hydrolysis of terminal non-reducing beta-D-glucose residues with the release of beta-D-glucose. Beta-glucosidase activity can be determined using pnitrophenyl-beta-D-glucopyranoside as substrate according to the procedure of Venturi et al., 2002, J. Basic Microbiol. 42: 55-66. One unit of beta-glucosidase is defined as 1.0 μmole of pnitrophenolate anion produced per minute at 25° C., pH 4.8 from 1 mM p-nitrophenyl-beta-Dglucopyranoside as substrate in 50 mM sodium citrate containing 0.01% TWEEN® 20.

Beta-xylosidase/polypeptide with beta-xylosidase activity: The term “beta-xylosidase” means a beta-D-xyloside xylohydrolase (E.C. 3.2.1.37) that catalyzes the exo-hydrolysis of short beta (1→4)-xylooligosaccharides to remove successive D-xylose residues from non-reducing termini. Beta-xylosidase activity can be determined using 1 mM p-nitrophenyl-beta-D-xyloside as substrate in 100 mM sodium citrate containing 0.01% TWEEN® 20 at pH 5, 40° C. One unit of beta-xylosidase is defined as 1.0 μmole of p15 nitrophenolate anion produced per minute at 40° C., pH 5 from 1 mM p-nitrophenyl-beta-Dxylosidein 100 mM sodium citrate containing 0.01% TWEEN® 20.

Cellobiohydrolase/polypeptide with cellobiohydrolase activity: The term “cellobiohydrolase” means a 1,4-beta-D-glucan cellobiohydrolase (E.C. 3.2.1.91 and E.C. 3.2.1.176) that catalyzes the hydrolysis of 1,4-beta-D15 glucosidic linkages in cellulose, cellooligosaccharides, or any beta-1,4-linked glucose containing polymer, releasing cellobiose from the reducing end (cellobiohydrolase I) or non-reducing end (cellobiohydrolase II) of the chain (Teeri, 1997, Trends in Biotechnology 15: 160-167; Teeri et al., 1998, Biochem. Soc. Trans. 26: 173-178). Cellobiohydrolase activity can be determined according to the procedures described by Lever et al., 1972, Anal. Biochem. 47: 273-279; van Tilbeurgh et al., 1982, FEBS Letters 149: 152-156; van Tilbeurgh and Claeyssens, 1985, FEBS Letters 187: 283-288; and Tomme et al., 1988, Eur. J. Biochem. 170: 575-581.

Cellulolytic enzyme or cellulase/polypeptide with cellulase activity or cellulolytic activity: The term “cellulolytic enzyme” or “cellulase” means one or more (e.g., several) enzymes that hydrolyze a cellulosic material, which comprise any material comprising cellulose, such as fiber. Cellulytic enzymes include endoglucanase(s) (E.C 3.2.1.4), cellobiohydrolase(s) (E.C 3.2.1.91 and E.C 3.2.1.150), beta-glucosidase(s) (E.C. 3.2.1.21), or combinations thereof. The two basic approaches for measuring cellulolytic enzyme activity include: (1) measuring the total cellulolytic enzyme activity, and (2) measuring the individual cellulolytic enzyme activities (endoglucanases, cellobiohydrolases, and beta-glucosidases) as reviewed in Zhang et al., 2006, Biotechnology Advances 24: 452-481. Total cellulolytic enzyme activity can be measured using insoluble substrates, including Whatman N^{o} 1 filter paper, microcrystalline cellulose, bacterial cellulose, algal cellulose, cotton, pretreated lignocellulose, etc. The most common total cellulolytic activity assay is the filter paper assay using Whatman N^{o} 1 filter paper as the substrate. The assay was established by the International Union of Pure and Applied Chemistry (IUPAC) (Ghose, 1987, Pure Appl. Chem. 59: 257-68).

Cellulolytic enzyme activity can be determined by measuring the increase in production/release of sugars during hydrolysis of a cellulosic material by cellulolytic enzyme(s) under the following conditions: 1-50 mg of cellulolytic enzyme protein/g of cellulose in pretreated corn stover (PCS) (or other pretreated cellulosic material) for 3-7 days at a suitable temperature such as 40° C.-80° C., e.g., 40° C., 45° C., 50° C., 55° C., 60° C., 65° C., 70° C., 75° C., or 80° C., and a suitable pH, such as 4-9, e.g., 4.0, 4.5, 5.0, 5.5, 6.0, 6.5, 7.0, 7.5, 8.0, 8.5, or 9.0, compared to a control hydrolysis without addition of cellulolytic enzyme protein. Typical conditions are 1 ml reactions, washed or unwashed PCS, 5% insoluble solids (dry weight), 50 mM sodium acetate 5 pH 5, 1 mM MnSO4, 50° C., 55° C., or 60° C., 72 hours, sugar analysis by AMINEX® HPX-87H column chromatography (Bio-Rad Laboratories, Inc., Hercules, Calif., USA).

Endoglucanase: The term “endoglucanase” means an endo-1,4-(1,3;1,4)-beta-D-glucan 4-glucanohydrolase (E.C. 3.2.1.4) that catalyzes endohydrolysis of 1,4-beta-D-glycosidic linkages in cellulose, cellulose derivatives (such as carboxymethyl cellulose and hydroxyethyl cellulose), lichenin, beta-1,4 bonds in mixed beta-1,3 glucans such as cereal beta-D-glucans or xyloglucans, and other plant material containing cellulosic components. Endoglucanase activity can be determined by measuring reduction in substrate viscosity or increase in reducing ends determined by a reducing sugar assay (Zhang et al., 2006, Biotechnology Advances 24: 452-481). For purposes of the present invention, endoglucanase activity is determined using carboxymethyl cellulose (CMC) as substrate according to the procedure of Ghose, 1987, Pure and Appl. Chem. 59: 257-268, at pH 5, 40° C.

Family 61 glycoside hydrolase: The term “Family 61 glycoside hydrolase” or “Family GH61” or “GH61” means a polypeptide falling into the glycoside hydrolase Family 61 according to Henrissat, 1991, A classification of glycosyl hydrolases based on amino-acid sequence similarities, Biochem. J. 280: 309-316, and Henrissat and Bairoch, 1996, Updating the sequence-based classification of glycosyl hydrolases, Biochem. J. 316: 695-696. The enzymes in this family were originally classified as a glycoside hydrolase family based on measurement of very weak endo-1,4-beta-D-glucanase activity in one family member. The structure and mode of action of these enzymes are non-canonical and they cannot be considered as bona fide glycosidases. However, they are kept in the CAZy classification on the basis of their capacity to enhance the breakdown of lignocellulose when used in conjunction with a cellulase or a mixture of cellulases. The GH61 polypeptides have recently been classified as lytic polysaccharide monooxygenases (Quinlan et al., 2011, Proc. Natl. Acad. Sci. USA 208: 15079-15084; Phillips et al., 2011, ACS Chem. Biol. 6: 1399-1406; Lin et al., 2012, Structure 20: 1051-1061) and are designated “Auxiliary Activity 9” or “AA9” polypeptides.

Hydrolytic enzymes or hydrolase/polypeptide with hydrolase activity: “Hydrolytic enzymes” refers to any catalytic protein that use water to break down substrates. Hydrolytic enzymes include cellulases (EC 3.2.1.4), xylanases (EC 3.2.1.8) arabinofuranosidases (EC 3.2.1.55 (Non-reducing end alpha-L-arabinofuranosidases); EC 3.2.1.185 (Non-reducing end beta-L-arabinofuranosidases) cellobiohydrolase I (EC 3.2.1.150), cellobiohydrolase II (E.C. 3.2.1.91), cellobiosidase (E.C. 3.2.1.176), beta-glucosidase (E.C. 3.2.1.21), beta-xylosidases (EC 3.2.1.37).

Xylanases/polypeptide with xylanase activity: The term “xylanase” means a 1,4-beta-D-xylan-xylohydrolase (E.C. 3.2.1.8) that catalyzes the endohydrolysis of 1,4-beta-D-xylosidic linkages in xylans. Xylanase activity can be determined with 0.2% AZCL-arabinoxylan as substrate in 0.01% TRITON® X-100 and 200 mM sodium phosphate pH 6 at 37° C. One unit of xylanase activity is defined as 1.0 μmole of azurine produced per minute at 37° C., pH 6 from 0.2% AZCL-arabinoxylan as substrate in 200 mM sodium phosphate pH 6. Xylanases can be found in, e.g., the GH5, GH30, GH10, and GH11 families.

GH5 polypeptide: refers to a polypeptide with enzyme activity, the polypeptide being classified as member of the Glycoside hydrolase family 5 in the database of Carbohydrate-Active EnZymes (CAZymes) (http://www.cazy.org/).

GH30 polypeptide: refers to a polypeptide with enzyme activity, the polypeptide being classified as member of the Glycoside hydrolase family 30 in the database of Carbohydrate-Active EnZymes (CAZymes) (http://www.cazy.org/).

GH10 polypeptide: refers to a polypeptide with enzyme activity, the polypeptide being classified as member of the Glycoside hydrolase family 10 in the database of Carbohydrate-Active EnZymes (CAZymes) available at http://www.cazy.org/. (Lombard, V.; Golaconda Ramulu, H.; Drula, E.; Coutinho, P. M.; Henrissat, B. (21 Nov. 2013). “The carbohydrate-active enzymes database (CAZy) in 2013”. Nucleic Acids Research. 42 (D1): D490-D495; Cantarel B L, Coutinho P M, Rancurel C, Bernard T, Lombard V, Henrissat B (January 2009). “The Carbohydrate-Active EnZymes database (CAZy): an expert resource for Glycogenomics”. Nucleic Acids Res. 37 (Database issue): D233-8).

GH11 polypeptide refers to a polypeptide with enzyme activity, the polypeptide being classified as member of the Glycoside hydrolase family 11 in the database of Carbohydrate-Active EnZymes (CAZymes).

GH62 polypeptide: refers to a polypeptide with enzyme activity, the polypeptide being classified as member of the Glycoside hydrolase family 62 in the database of Carbohydrate-Active EnZymes (CAZymes).

GH43 polypeptide: refers to a polypeptide with enzyme activity, the polypeptide being classified as member of the Glycoside hydrolase family 62 in the database of Carbohydrate-Active EnZymes (CAZymes).

### Other Definitions

Corn kernel: A variety of corn kernels are known, including, e.g., dent corn, flint corn, pod corn, striped maize, sweet corn, waxy corn and the like.

Some corn kernels has an outer covering referred to as the “Pericarp” that protects the germ in the kernels. It resists water and water vapour and is undesirable to insects and microorganisms. The only area of the kernels not covered by the “Pericarp” is the “Tip Cap”, which is the attachment point of the kernel to the cob.

Corn kernel mass: is preferably used to reference a mass comprising fiber, gluten and starch, preferably achieved by steaming and grinding crop kernels and separating a mass comprising fiber, gluten and starch from germs. As the corn kernel mass move through the fiber washing, it is separated into several fractions, including first (s) and second fractions (f). Hence, “fractions of corn kernel mass” and “one or more fractions of corn kernel mass” refer to these first (s) and second fractions (f).

Dewatering: “Dewatering” refers to any process in which excess water is removed from corn fiber.

Germ: The “Germ” is the only living part of the corn kernel. It contains the essential genetic information, enzymes, vitamins, and minerals for the kernel to grow into a corn plant. In yellow dent corn, about 25 percent of the germ is corn oil. The endosperm covered or surrounded by the germ comprises about 82 percent of the kernel dry weight and is the source of energy (starch) and protein for the germinating seed. There are two types of endosperm, soft and hard. In the hard endosperm, starch is packed tightly together. In the soft endosperm, the starch is loose.

Gluten: Gluten is a protein, made up from two smaller proteins, glutenin and gliadin. Herein “gluten” refers to the majority of proteins found in corn kernels. The major products of gluten from corn wet milling is corn gluten meal (Approximately 60% protein) and corn gluten feed (Approximately 20% protein).

Grind or grinding: The term “grinding” refers to breaking down the corn kernels into smaller components.

Insolubles: In the present context, “insolubles” is used interchangeably with “insoluble solids”; it is defined as materials that is able to pass through a 75 μm sieve and cannot be dissolved in water.

Mill equipment: “Mill equipment” refers to all equipment used on a mill. The wet milling process will vary dependent on the available mill equipment. Examples of mill equipment can be steeping tanks, evaporator, screw press, rotatory dryer, dewatering screen, centrifuge, hydrocyclone, ect. The size, and number of each mill equipment/milling lines can vary on different mills, which will affect the milling process. For example, the number of fiber washing screen units can vary and so can the size of a centrifuge.

Screened: The term “screened” refers to the process of separating corn kernel mass into a first fraction s and a second fraction f and movement of these fractions from one screen unit to another. A screen unit may for example be a pressure-fed screen/feed pressure screen wherein material is fed through a nozzle or a rotary screen, wherein material is forced through the screen by gravity. Examples of such screens could be DSM screen and ICM screens respectively.

A non-screening period is a non-separating period provided for incubation of corn kernel mass or fractions thereof with enzymes.

Sequence identity: The relatedness between two amino acid sequences or between two nucleotide sequences is described by the parameter “sequence identity”.

For purposes of the present invention, the degree of sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 3.0.0 or later. Version 6.1.0 was used.

The optional parameters used are gap open penalty of 10, gap extension penalty of 0.5, and the EBLOSUM62 (EMBOSS version of BLOSUM62) substitution matrix. The output of Needle labelled “longest identity” (obtained using the −nobrief option) is used as the percent identity and is calculated as follows: (Identical Residues×100)/(Length of Alignment−Total Number of Gaps in Alignment).

Starch: The term “starch” means any material comprised of complex polysaccharides of plants, composed of glucose units that occurs widely in plant tissues in the form of storage granules, consisting of amylose and amylopectin, and represented as (C₆H₁₀O₅)n, where n is any number.

Steeping or soaking: The term “steeping” means soaking the crop kernel with water and optionally SO₂.

### DETAILED DESCRIPTION

It is an object of the present invention to provide a method that improves oil yield from corn germ in a wet milling process.

The Wet Milling Process:

Corn kernels are wet milled in order to open up the kernels and separate the kernels into its four main constituents: starch, germ, fiber and gluten.

The wet milling process can vary significantly from mill to mill, however conventional wet milling usually comprises the following steps:

1. Steeping

2. Grinding

3. Separation into streams comprising:
i) germ; ii) fiber, iii) starch and gluten

4. Fiber washing, pressing and drying

5. Starch/gluten separation, and

6. Starch washing.

Steeping, Grinding and Germ Separation

Corn kernels are softened by soaking in water for between about 30 minutes to about 48 hours, preferably 30 minutes to about 15 hours, such as about 1 hour to about 6 hours at a temperature of about 50° C., such as between about 45° C. to 60° C. During steeping, the kernels absorb water, increasing their moisture levels from 15 percent to 45 percent and more than doubling in size. The optional addition of e.g. 0.1 percent sulphur dioxide (SO₂) and/or NaHSO₃ to the water prevents excessive bacteria growth in the warm environment. As the corn swells and softens, the mild acidity of the steep water begins to loosen the gluten bonds within the corn and release the starch. After the corn kernels are steeped they are cracked open to release the germ. The germ contains corn oil. The germ is separated from the heavier density mixture of starch, gluten and fiber essentially by “floating” the germ segment free of the other substances under closely controlled conditions. This method serves to eliminate any adverse effect of traces of corn oil in later processing steps.

Fiber Washing, Pressing and Drying

To get maximum starch and gluten recovery, while keeping any fiber in the final product to an absolute minimum, it is necessary to wash the free starch and gluten from the fiber during processing. The free starch and gluten is separated from fiber during screening (washing) and collected as mill starch. The remaining fiber is then pressed to decrease the

Starch Gluten Separation

The starch-gluten suspension from the fiber-washing step, called mill starch, is separated into starch and gluten. Gluten has a low density compared to starch. By passing mill starch through a centrifuge, the gluten is readily spun out.

Starch Washing

The starch slurry from the starch separation step contains some insoluble protein and much of solubles. They have to be removed before a top quality starch (high purity starch) can be made. The starch, with just one or two percent protein remaining, is diluted, washed 8 to 14 times, re-diluted and washed again in hydro-clones to remove the last trace of protein and produce high quality starch, typically more than 99.5% pure.

Products of wet milling: Wet milling can be used to produce, without limitation, corn steep liquor, corn gluten feed, germ, corn oil, corn gluten meal, corn starch, modified corn starch, syrups such as corn syrup, and corn ethanol.

In processes for conventional corn wet milling, grinding is followed by germ separation (germ is separated from starch/gluten and fiber) and germ drying. Oil can then be extracted from dried germs.

The present inventors have observed that contacting/incubating the germ with one or more xylanases will result in increased oil extraction yields from the germ.

Thus, in a first aspect the present invention relates to a method for improving the oil yield from germ in a wet milling process, the method comprising admixing a process stream comprising germ with an enzyme composition comprising an effective amount of one or more hydrolytic enzymes, wherein at least one of said hydrolytic enzymes is a xylanase polypeptide selected from the group consisting of: GH5, GH10, GH30, GH11 polypeptides.

In one embodiment the oil released from the germ according to the method of the invention is increase compared to a process where no enzymatic treatment occurs.

More particularly the present invention relates to a wet-milling method comprising the steps of:
a) soaking corn kernels in water to produce soaked kernels;
b) grinding the soaked kernels to release germ from kernels;
c) separating germs from the soaked and ground kernels; and
d) subjecting the germ to enzymatic treatment with at least one xylanase polypeptide selected from the group consisting of: GH5, GH10, GH30, GH11 xylanases.

In one embodiment the said germ is admixed with said one or more xylanase enzymes during or after step b) according to the invention, preferably during step c) or after the germs have been separated.

In a particular embodiment said germ is admixed with said one or more xylanase enzymes after step c)

The enzymatic treatment should be allowed to proceed for a sufficient time and with and effective amount. The skilled person will be able to determine this depending on the wet-milling conditions and the specific enzymes applied. In one embodiment the germ is allowed to react with said one or more xylanase enzymes for at least 45 min, e.g., at least 1 hour, such as at least 2 hours, such as at least 2.5 hours, such as at least 3 hours. Preferably, the xylanase polypeptide is present in an amount of preferably 0.0005 to 1.5 mg enzyme protein per g DS kernels, preferably 0.001 to 1 mg enzyme protein per g DS kernels, preferably 0.002 to 0.5 mg enzyme protein per g DS kernels, preferably 0.003 to 0.4 mg enzyme protein per g DS kernels.

In one embodiment the hydrolytic enzymes comprises at least one xylanase and at least one arabinofuranosidase. The arabinofuranosidase is a GH62 or a GH43 arabinofuranosidase, preferably a GH62 arabinofuranosidase.

In one embodiment the hydrolytic enzymes comprises at least one beta-glucanase. The beta-glucanase is in one embodiment a GH5 beta-glucanase, particularly a GH5_15 beta-glucanase.

In another embodiment the xylanase enzymes is at least one GH5 xylanase, particularly a GH5_21 xylanase.

In another embodiment the xylanase is a is a GH10 xylanase.

In another embodiment the xylanase is a is a GH11 xylanase.

In another embodiment the xylanase is a is a GH30 xylanase, particularly a GH30_8 xylanase.

The hydrolytic enzymes may further comprise cellulases. E.g., the xylanase and/or arabinofuranosidase may be expressed in a Trichoderma host organism, particularly a Trichoderma reesei host organism and the cellulases produced by the host may be include in the enzyme composition.

Irrespective of which filamentous fungal organisms may be used as the source of cellulases, it is preferred that the cellulases are selected from at least endoglucanase(s) and a cellobiohydrolase(s). Particularly, the cellulases may be selected from endoglucanases (EG), cellobiohydrolases I (CBH I), cellobiohydrolases II (CBH II), GH61, beta-glucosidases, or a combination thereof.

In one embodiment the cellulases may comprise at least CBH I, CBH II, and EG I.

In a specific embodiment the xylanase is selected from the group consisting of:
(a) a polypeptide having at least 75%, at least 80%, 85%, e.g., at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 1;
(b) a variant of the mature polypeptide of SEQ ID NO: 1 comprising a substitution, deletion, and/or insertion at one or several positions; and
(c) a fragment of the polypeptide of (a), or (b) that has xylanase activity;
and wherein preferably the xylanase of a), b), and c) have at least 20%, e.g., at least 40%, at least 50%, at least 60%, at least 70%, at least 80%, at least 90%, at least 95%, or at least 100% of the xylanase activity of the mature polypeptide of SEQ ID NO: 1.

The mature polypeptide is in one embodiment amino acids 21 to 405 of SEQ ID NO: 1.

In another specific embodiment the arabinofuranosidase is selected from the group consisting of:
(a) a polypeptide having at least 75%, at least 80%, 85%, e.g., at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 2;
(b) a variant of the mature polypeptide of SEQ ID NO: 2 comprising a substitution, deletion, and/or insertion at one or several positions; and
(c) a fragment of the polypeptide of (a), or (b) that has arabinofuranosidase activity; and wherein preferably the arabinofuranosidase of a), b) and c) have at least 20%, e.g., at least 40%, at least 50%, at least 60%, at least 70%, at least 80%, at least 90%, at least 95%, or at least 100% of the arabinofuranosidase activity of the mature polypeptide of SEQ ID NO: 2.

The mature polypeptide is in one embodiment amino acids 17 to 325 of SEQ ID NO: 2.

In another specific embodiment the xylanase is selected from the group consisting of:
(a) a polypeptide having at least 75%, at least 80%, 85%, e.g., at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 4;
(b) a variant of the mature polypeptide of SEQ ID NO: 4 comprising a substitution, deletion, and/or insertion at one or several positions; and
(c) a fragment of the polypeptide of (a), or (b) that has xylanase activity;
   and wherein preferably the xylanase of a), b), and c) have at least 20%, e.g., at least 40%, at least 50%, at least 60%, at least 70%, at least 80%, at least 90%, at least 95%, or at least 100% of the xylanase activity of the mature polypeptide of SEQ ID NO: 4.

The mature polypeptide is in one embodiment amino acids 1 to 551 of SEQ ID NO: 4.

In another specific embodiment the xylanase is selected from the group consisting of:
(a) a polypeptide having at least 75%, at least 80%, 85%, e.g., at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 5;
(b) a variant of the mature polypeptide of SEQ ID NO: 5 comprising a substitution, deletion, and/or insertion at one or several positions; and
(c) a fragment of the polypeptide of (a), or (b) that has xylanase activity;
   and wherein preferably the xylanase of a), b), and c) have at least 20%, e.g., at least 40%, at least 50%, at least 60%, at least 70%, at least 80%, at least 90%, at least 95%, or at least 100% of the xylanase activity of the mature polypeptide of SEQ ID NO: 5.

The mature polypeptide is in one embodiment amino acids 28 to 417 of SEQ ID NO: 5.

In another specific embodiment the xylanase is selected from the group consisting of:
(a) a polypeptide having at least 75%, at least 80%, 85%, e.g., at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 6;
(b) a variant of the mature polypeptide of SEQ ID NO: 6 comprising a substitution, deletion, and/or insertion at one or several positions; and
(c) a fragment of the polypeptide of (a), or (b) that has xylanase activity;
   and wherein preferably the xylanase of a), b), and c) have at least 20%, e.g., at least 40%, at least 50%, at least 60%, at least 70%, at least 80%, at least 90%, at least 95%, or at least 100% of the xylanase activity of the mature polypeptide of SEQ ID NO: 6.

The mature polypeptide is in one embodiment amino acids 30 to 212 of SEQ ID NO: 6.

In another specific embodiment the beta-glucanase is selected from the group consisting of:
(a) a polypeptide having at least 75%, at least 80%, 85%, e.g., at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 7;
(b) a variant of the mature polypeptide of SEQ ID NO: 7 comprising a substitution, deletion, and/or insertion at one or several positions; and
(c) a fragment of the polypeptide of (a), or (b) that has beta-glucanase activity;
   and wherein preferably the beta-glucanase of a), b), and c) have at least 20%, e.g., at least 40%, at least 50%, at least 60%, at least 70%, at least 80%, at least 90%, at least 95%, or at least 100% of the beta-glucanase activity of the mature polypeptide of SEQ ID NO: 7.

The mature polypeptide is in one embodiment amino acids 17 to 408 of SEQ ID NO: 7.

In a particular embodiment of the invention the germ is dewatered/dried before being contacted with the enzymes.

A further embodiment of the claimed method comprises the step of extracting oil from the germ.

The present invention is further disclosed in the following numbered paragraphs.

Paragraph 1. A method for improving oil yield from germ in a wet milling process, the method comprising admixing a process stream comprising corn germ with an enzyme composition comprising an effective amount of one or more hydrolytic enzymes, wherein at least one of said hydrolytic enzymes is at least one xylanase polypeptide selected from the group consisting of: GH5, GH10, GH30, GH11 polypeptides.

Paragraph 2. The method according to paragraph 1, wherein the amount of oil released from the germ is increased compared to no xylanase enzyme addition.

Paragraph 3. The method according to any of the preceding paragraphs, comprising the steps of:
a) soaking the corn kernels in water to produce soaked kernels;
b) grinding the soaked kernels to release germ from kernels;
c) separating germs from the soaked and ground kernels; and
   subjecting the germ to enzymatic treatment with at least one xylanase polypeptide selected from the group consisting of: GH5, GH10, GH30, GH11 xylanases.

Paragraph 4. The method according to any of the preceding paragraphs, wherein said germ is admixed with said one or more xylanase enzymes during or after step b) according to paragraph 3, preferably during step c) or after the germs have been separated.

Paragraph 5. The method according to any of the preceding paragraphs, wherein said germ is admixed with said one or more xylanase enzymes after step c) according to paragraph 3.

Paragraph 6. The method according to any of the preceding paragraphs, wherein said germ is allowed to react with said one or more xylanase enzymes for at least 45 min, at least 1 hour, such as at least 2 hours, such as at least 2.5 hours, such as at least 3 hours.

Paragraph 7. The method of any of the preceding paragraphs, wherein said hydrolytic enzyme is present in an amount of preferably 0.0005 to 1.5 mg enzyme protein per g DS kernels, preferably 0.001 to 1 mg enzyme protein per g DS kernels, preferably 0.002 to 0.5 mg enzyme protein per g DS kernels, preferably 0.003 to 0.4 mg enzyme protein per g DS kernels.

Paragraph 8. The method according to any of the preceding paragraphs wherein the hydrolytic enzymes comprises at least one xylanase and at least one arabinofuranosidase.

Paragraph 9. The method according to any of the preceding paragraphs, wherein the hydrolytic enzymes comprises at least one beta-glucanase.

Paragraph 10. The method according to any of the preceding paragraphs, wherein the hydrolytic enzymes further comprise cellulases.

Paragraph 11. The method of paragraph 9, wherein the cellulases are selected from endoglucanases, cellobiohydrolases I, cellobiohydrolases II, GH61, beta-glucosidases, or a combination thereof.

Paragraph 12. The method of paragraph 11, wherein the cellulases comprise at least an endoglucanase and a cellobiohydrolase.

Paragraph 13. The method of paragraph 12, wherein the cellulases comprise at least cellobiohydrolases I, cellobiohydrolases II, and endoglucanase I.

Paragraph 14. The method of paragraphs 9-13, wherein the cellulases are derived from Trichoderma, particularly Trichoderma reesei.

Paragraph 15. The method according to any of the preceding paragraphs, wherein the xylanase is selected from Glycosyl Hydrolase family GH5, GH30, GH10, GH11.

Paragraph 16. The method according to paragraph 15, wherein the xylanase is a GH10 xylanase.

Paragraph 17. The method according to paragraph 15, wherein the xylanase is a GH5 xylanase, particularly GH5_21 xylanas.

Paragraph 18. The method according to paragraph 15, wherein the xylanase is a GH11 xylanase.

Paragraph 19. The method according to paragraph 15, wherein the xylanase is a GH30 xylanase, particularly a GH30_8 xylanase.

Paragraph 20. The method according to paragraphs 8-19, wherein the arabinofuranosidase is a GH62 or a GH43 arabinofuranosidase.

Paragraph 21. The method according to any of the paragraphs 9-20, wherein the beta-glucanase is a GH5 beta-glucanase, particularly a GH5_15 beta-glucanase.

Paragraph 22. The method according to any of the preceding paragraphs wherein the xylanase is selected from the group consisting of:
(a) a polypeptide having at least 75%, 80%, 85%, e.g., at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 1;
(b) a variant of the mature polypeptide of SEQ ID NO: 1 comprising a substitution, deletion, and/or insertion at one or several positions; and
(c) a fragment of the polypeptide of (a), or (b) that has xylanase activity;
and wherein preferably the xylanase of a), b), and c) have at least 20%, e.g., at least 40%, at least 50%, at least 60%, at least 70%, at least 80%, at least 90%, at least 95%, or at least 100% of the xylanase activity of the mature polypeptide of SEQ ID NO: 1.

Paragraph 23. The method of paragraph 22, wherein the mature polypeptide is amino acids 21 to 405 of SEQ ID NO: 1.

Paragraph 24. The method of any of the paragraphs 8-23, wherein the arabinofuranosidase is selected from the group consisting of:
(a) a polypeptide having at least 75%, at least 80%, 85%, e.g., at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 2;
(b) a variant of the mature polypeptide of SEQ ID NO: 2 comprising a substitution, deletion, and/or insertion at one or several positions; and
(c) a fragment of the polypeptide of (a), or (b) that has arabinofuranosidase activity;
and wherein preferably the arabinofuranosidase of a), b) and c) have at least 20%, e.g., at least 40%, at least 50%, at least 60%, at least 70%, at least 80%, at least 90%, at least 95%, or at least 100% of the arabinofuranosidase activity of the mature polypeptide of SEQ ID NO: 2.

Paragraph 25. The method of paragraph 24, wherein the mature polypeptide is amino acids 17 to 325 of SEQ ID NO: 2.

Paragraph 26. The method according to any of the preceding paragraphs wherein the xylanase is selected from the group consisting of:
(a) a polypeptide having at least 75%, at least 80%, 85%, e.g., at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 4;
(b) a variant of the mature polypeptide of SEQ ID NO: 4 comprising a substitution, deletion, and/or insertion at one or several positions; and
(c) a fragment of the polypeptide of (a), or (b) that has xylanase activity;
and wherein preferably the xylanase of a), b), and c) have at least 20%, e.g., at least 40%, at least 50%, at least 60%, at least 70%, at least 80%, at least 90%, at least 95%, or at least 100% of the xylanase activity of the mature polypeptide of SEQ ID NO: 4.

Paragraph 27. The method of paragraph 26, wherein the mature polypeptide is amino acids 1 to 551 of SEQ ID NO: 4.

Paragraph 28. The method according to any of the preceding paragraphs wherein the xylanase is selected from the group consisting of:
(a) a polypeptide having at least 75%, at least 80%, 85%, e.g., at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 5;
(b) a variant of the mature polypeptide of SEQ ID NO: 5 comprising a substitution, deletion, and/or insertion at one or several positions; and
(c) a fragment of the polypeptide of (a), or (b) that has xylanase activity;
and wherein preferably the xylanase of a), b), and c) have at least 20%, e.g., at least 40%, at least 50%, at least 60%, at least 70%, at least 80%, at least 90%, at least 95%, or at least 100% of the xylanase activity of the mature polypeptide of SEQ ID NO: 5.

Paragraph 29. The method of paragraph 28, wherein the mature polypeptide is amino acids 28 to 417 of SEQ ID NO: 5.

Paragraph 30. The method according to any of the preceding paragraphs wherein the xylanase is selected from the group consisting of:
(a) a polypeptide having at least 75%, at least 80%, 85%, e.g., at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 6;
(b) a variant of the mature polypeptide of SEQ ID NO: 6 comprising a substitution, deletion, and/or insertion at one or several positions; and
(c) a fragment of the polypeptide of (a), or (b) that has xylanase activity;
and wherein preferably the xylanase of a), b), and c) have at least 20%, e.g., at least 40%, at least 50%, at least 60%, at least 70%, at least 80%, at least 90%, at least 95%, or at least 100% of the xylanase activity of the mature polypeptide of SEQ ID NO: 6.

Paragraph 31. The method of paragraph 30, wherein the mature polypeptide is amino acids 30 to 212 of SEQ ID NO: 6.

Paragraph 32. The method according to any of paragraphs 9-31, wherein the beta-glucanase is selected from the group consisting of:
(a) a polypeptide having at least 75%, at least 80%, 85%, e.g., at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of SEQ ID NO: 7;
(b) a variant of the mature polypeptide of SEQ ID NO: 7 comprising a substitution, deletion, and/or insertion at one or several positions; and
(c) a fragment of the polypeptide of (a), or (b) that has beta-glucanase activity;
and wherein preferably the beta-glucanase of a), b), and c) have at least 20%, e.g., at least 40%, at least 50%, at least 60%, at least 70%, at least 80%, at least 90%, at least 95%, or at least 100% of the beta-glucanase activity of the mature polypeptide of SEQ ID NO: 7.

Paragraph 33. The method of paragraph 32, wherein the mature polypeptide is amino acids 17 to 408 of SEQ ID NO: 7.

Paragraph 34. The method according to any of the preceding paragraphs, wherein the germ is dewatered/dried.

Paragraph 35. The method according to any of the preceding paragraphs further comprising the step of extracting oil from the germ.

The invention is further illustrated in the following examples.

### EXAMPLES

Enzymes Used in the Examples

GH10 xylanase from Talaromyces leycettanus. Disclosed herein as SEQ ID NO: 1 (mature polypeptide amino acids 21-405)

GH62 arabinofuranosidase from Talaromyces phinophilus. Disclosed herein as SEQ ID NO: 2 (mature polypeptide amino acids 17-325).

Metalloprotease from Thermoascus aurantiacus. Disclosed herein as SEQ ID NO: 3 (mature polypeptide amino acids 1-177).

GH5_21 xylanase from Chryseobacterium sp. Disclosed herein as SEQ ID NO: 4 (mature polypeptide amino acids 1-551).

GH30_8 xylanase from Bacillus sp-18423. Disclosed herein as SEQ ID NO: 5 (mature polypeptide amino acids 28-417).

GH11 xylanase from Geobacillus stearothermophilus. Disclosed herein as SEQ ID NO: 6 (mature polypeptide amino acids 30-212).

GH5_15 endo-1,6-beta glucanase from Trichoderma astroviride. Disclosed herein as SEQ ID NO: 7 (mature polypeptide amino acids 17-408).

Beta-Glucanase Activity Assays

Beta-glucanase activity is determined by measuring concentration of reducing sugars (RS) released by a beta-glucanase after hydrolysis of appropriate beta-glucan substrate. Activity of GH16 beta-1,3(4)-glucanases and GH64 beta-1,3-glucanases is determined using CM-Pachyman (beta-1,3-glucan, P-CMPAC, Megazyme). Activity of GH5_15 beta-1,6-glucanases and GH30_3 beta-1,6-glucanases is determined using Pustulan (beta-1,6-glucan, YP15423, Carbosynth). The RS concentration is measured using p-hydroxybenzoic acid hydrazide (PHBAH) assay adapted to a 96-well microplate format. In the assay, the reaction between reducing ends of C6 and C5 sugars and PHBAH results in a formation of hydrazones, which have intense yellow color and can be detected by absorbance measurement at 410 nm.

Enzymatic Hydrolysis of Beta-Glucan Substrate

Enzymatic hydrolysis is initiated by combining 80 ul of 2.5 g/L beta-glucan substrate, 10 ul of appropriately diluted enzyme sample, and 10 ul of 50 mM Glucono-Delta-Lactone (GDL) in a hard-shell 96-well PCR plate (HSP-9631, Bio-Rad). GDL is added to inhibit beta-glucosidase activity in an expression host background. Each incubation mixture (total volume 100 ul) includes 2 g/L substrate, enzyme, and 5 mM GDL in 50 mM Na-Acetate buffer, pH 5.0. The plate is sealed with an aluminum sealing tape (Costar #6570, Corning Inc.), and incubated in a thermocycler (Mastercycler Pro S, Eppendorf) at 50° C. for 10 min, followed by cooling down to 10° C. Each enzyme sample is serially diluted 2-fold eight times in 50 mM sodium acetate buffer, pH 5.0 to generate protein dose profile, and each enzyme dose is typically assayed in triplicate. Each plate includes two sets of glucose standards, 0.0625-1 mM and 0.3125-5 mM. Glucose standards are prepared by diluting 10 mM stock glucose solution in 50 mM sodium acetate buffer, pH 5.0. Each glucose standard (100 ul) is treated similarly to the samples.

### Example 1: Oil Extraction Yields from Corn Germ after First Wind Using GH10 Xylanase and GH62 Arabinofuranosidase

Substrate and experimentation preparation: Industrially generated de-watered wet milled germ from corn kernels was used as the substrate for experimentation. The xylanase used was a GH10 xylanase disclosed herein as SEQ ID NO: 1 and the GH62 arabinofuranosidase was disclosed herein as SEQ ID NO: 2. The xylanase and the arabinofuranosidase were expressed in Trichoderma reesei in two separate fermentations and subsequently mixed. Thus, the mixture contained all cellulases normally expressed in Trichoderma reesei.

A dry solids measurement of 51.79% was observed on the substrate. The dry solids measurement was generated from a moisture balance. An amount of roughly 200 grams of germ was weighed out to give a 0.1 dry solids per flask. An enzyme concentration of 10 g enzyme product/kg DS was used as a dose. Use of xylanase and arabinofuranosidase was compared to the known process of adding protease in order to increase oil yield. The protease used was a wild type protease derived from Thermoascus aurantiacus disclosed in WO 2003/048353 and herein as SEQ ID NO: 3. The flask containing germ and enzyme were adjusted to a final dry solids of 2.6% with tap water.

TABLE 1
Sample set-up

| | Germ | Germ | | | | Water |
|---|---|---|---|---|---|---|
| | weight | DS | | | | added |
| Treatment | (kg) | (kg) | ml | ml | % DS | (ml) |
| — | 0.20114 | 0.10 | 0 | 0 | 2.60% | 400 |
| — | 0.20158 | 0.10 | 0 | 0 | 2.60% | 401 |
| — | 0.2016 | 0.10 | 0 | 0 | 2.60% | 401 |
| SEQ ID | 0.20107 | 0.10 | 0.85 | 0 | 2.60% | 399 |
| NO: 1 + 2 | | | | | | |
| SEQ ID | 0.2026 | 0.10 | 0.86 | 0 | 2.60% | 402 |
| NO: 1 + 2 | | | | | | |
| SEQ ID | 0.2016 | 0.10 | 0.86 | 0 | 2.60% | 401 |
| NO: 1 + 2 | | | | | | |
| SEQ ID | 0.20516 | 0.11 | 0 | 0.89 | 2.60% | 408 |
| NO: 3 | | | | | | |
| SEQ ID | 0.20163 | 0.10 | 0 | 0.87 | 2.60% | 401 |
| NO: 3 | | | | | | |
| SEQ ID | 0.20467 | 0.11 | 0 | 0.88 | 2.60% | 407 |
| NO: 3 | | | | | | |

The flask was incubated at 48 degrees Celsius for 24 hours with constant mixing. After 24 hours of incubation the germ slurry was poured over a filtrate funnel to allow liquid portion to flow through and only germ solids were left. Once the germ was dewatered it was placed in an oven at 50 degrees Celsius to facilitate drying.

Data Measurement: The data generated was according to the official Methods of Analysis (http://www.eoma.aoac.org/).
Protein (crude) AOAC 990.03
Fat (acid hydrolysis) AOAC 954.02
Fiber (crude) AOCS Ba 6a-05
   Analysis followed MWL FD 039 which is based on AOCS Ba 6a-05. A small amount of sample was weighed and placed in a membrane bag and sealed. The bag and sample were placed in a container that treats the sample with a variety of chemicals to dissolve materials which leach out of the bag. After repeated washing and rinsing, the bag was dried and reweighed. The material remaining in the bag was reported as crude fiber.

TABLE 2
Analysis data

| | | % Fat (Acid | |
|---|---|---|---|
| Treatment | % Protein | Hydrolysis) | % Fiber |
| Control no | 15.4 | 42.6 | 9.86 |
| incubation | | | |
| Control | 14.6 | 43.2 | 6.49 |
| Incubation | | | |
| SEQ ID | 13.4 | 47 | 4.51 |
| NO 1 + | | | |
| SEQ ID | | | |
| NO: 2 | | | |
| SEQ ID | 12.9 | 42.9 | 7.1 |
| NO: 3 | | | |

Oil Extraction:

Dried corn germ was placed in a hopper of a pressing equipment. The equipment has a screw that acts as a press and squeezes the oil out of the germ. Oil flows though the slots under the screw and is collected in a beaker or suitable container.

A mass measurement was taken on the oil that was recovered. Oil recovery was determined based on the mass measurement. The amount of germ going into the hopper was recorded and based on the difference between oil mass and germ mass, the effectiveness of the enzyme was evaluated. Results for effectiveness of oil recovery is shown in Table 3 below.

TABLE 3

| Oil recovery | Total Dry | Oil |
|---|---|---|
| Treatment | Substance (g) | Recovered |
| Incoming Ingredion Flaked | 29.49 | 9.59 |
| Germ (de-watered) | | |
| Control No Enzyme | 29.83 | 10.21 |
| SEQ ID NO: 1 + 2 | 29.76 | 11.82 |
| SEQ ID NO: 3 | 29.84 | 10.29 |

### Example 2. Comparative Test of Further Xylanase Diversity According to the Method of the Invention

Germ Washing with Xylanase Enzymes

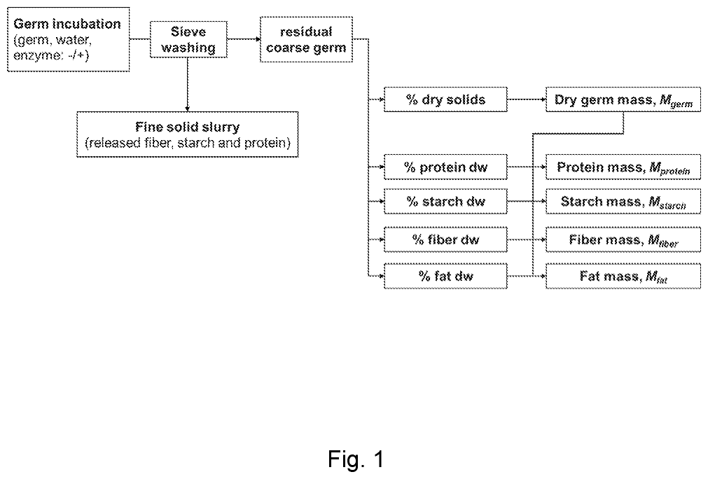

Five enzymes belonging to GH enzyme family groups: GH5_15, GH5_21, GH10, GH11 and GH30_8 was tested for germ treatments. Of these, GH5_15 is a beta-glucanase while the remaining four were xylanase enzymes. Background cellulase enzyme was also present in each of the enzyme treatment. The background cellulases were obtained as all cellulases produced from a strain of Trichoderma reesei Tv30. As illustrated in FIG. 1, enzyme treatment comprised of incubating 1 L corn germ slurry, at 7.3% DS: 136 g of corn germ sample (moisture content: 46.4%), incubated with 760 ug total enzyme protein per gram dry solids at 48 degree Celsius and pH 4 for 4 hours. A control: no enzyme treatment was also included for comparison. After incubation, germ was washed with 2 L of water over a 75 μm screen to separate coarse germ from fine solids. The germ samples were oven dried and analyzed for crude fiber, protein, and fat content via standardized AOCS (American Oil Chemists' Society) methods: Ba 6a-05, 990.03 and 2003.05 respectively. Starch analysis was conducted via dilute acid hydrolysis method. Baseline (no enzyme) treatment of germ sample had: 16.6% crude fiber, 8.4% starch, 10.9% crude protein and 44.1% crude fat on dry weight basis.

Mass of each analyte in respective washed germ samples were calculated as:

Mass of analyte=mass of dried germ×% dw of analyte

For example,

Mass of protein, Mₚᵣₒₜₑᵢₙ=M_{germ}×% protein dw

For enzyme treatment, reduction of an analyte mass was calculated as:

% reduction of analyte mass=[Analyte mass (enzyme treated−control)/analyte mass of control]×100%

For example, percent reduction of germ protein mass for an enzyme treatment is relative difference of protein mass for enzyme treated and control germ with respect to protein mass of control germ. Illustratively,

% reduction of germ protein mass=Mₚᵣₒₜₑᵢₙ of (enzyme treated−control)/Mₚᵣₒₜₑᵢₙ of control germ×100%

The enzyme treatments germ liberated and separated fiber, starch, and protein into fine solid slurry fraction. Table 4 shows percent reduction of germ, fiber, starch, protein, and fat mass for residual coarse germ via various enzyme treatments with respect to no enzyme as control treatment. With enzyme treatments, considerable amount of fiber, starch and protein were liberated during incubation and later separated into fine solid slurry fractions. Recovery of liberated starch and protein from fine solid slurry in downstream corn wet-milling process would increase the yields of these co-products.

Minimal change in crude fat mass was observed with enzyme treatments. Removal of fiber, starch and protein into fine solid slurry fraction enriched the proportion of crude fat in enzyme treated germ sample as shown in table 5. Therefore, oil yield of enzyme treated germ samples will also be improved.

TABLE 4
Percent reduction in dry mass, crude fiber, starch, protein,
and fat in residual coarse germ after enzyme treatment

| Enzyme used | | Crude | | Crude | Crude |
|---|---|---|---|---|---|
| for germ | Dry | fiber | Starch | protein | fat |
| incubation | mass | mass | mass | mass | mass |
| No enzyme | 0.0% | 0.0% | 0.0% | 0.0% | 0% |
| GH5_21 | 21.3% | 66.6% | 4.6% | 17.7% | 5% |
| SEQ ID NO: 4 | | | | | |
| GH30_8 | 12.5% | 60.0% | 6.8% | 6.9% | −1% |
| SEQ ID NO: 5 | | | | | |
| GH11 | 7.3% | 22.4% | −0.8% | 8.2% | −3% |
| SEQ ID NO: 6 | | | | | |
| GH5_15 | 15.9% | 58.5% | 20.8% | 12.1% | 1% |
| SEQ ID NO: 7 | | | | | |
| GH10 | 9.6% | 70.8% | 14.5% | 2.2% | −1% |
| SEQ ID NO: 1 | | | | | |
| GH5_15/GH10 | 7.5% | 65.3% | −3.4% | 5.9% | −4% |
| SEQ ID NO: 7 + 1 | | | | | |

TABLE 5
Percent crude fat in residual coarse germ after enzyme treatment

| Enzyme used for | Crude fat % of |
|---|---|
| germ incubation | enzyme treated germ |
| No enzyme | 44.1% |
| GH5_21 | 53.4% |
| SEQ ID NO: 4 | |
| GH30_8 | 51.0% |
| SEQ ID NO: 5 | |
| GH11 | 49.2% |
| SEQ ID NO: 6 | |
| GH5_15 | 46.4% |
| SEQ ID NO: 7 | |
| GH10 | 51.8% |
| SEQ ID NO: 1 | |
| GH5_15/GH10 | 49.1% |
| SEQ ID NO: 7 + 1 | |

1. A method for improving oil yield from germ in a wet milling process, the method comprising admixing a process stream comprising corn germ with an enzyme composition comprising an effective amount of one or more hydrolytic enzymes, wherein at least one of said hydrolytic enzymes is a xylanase selected from the group consisting of a GH5 xylanase, a GH10 xylanase, a GH30 xylanase, and a GH11 xylanase.

2. The method according to claim 1, wherein the amount of oil released from the germ is increased compared to the amount of oil released without the xylanase.

3. The method according to claim 2, comprising the steps of:
a) soaking the corn kernels in water to produce soaked kernels;
b) grinding the soaked kernels to release germ from kernels;
c) separating germs from the soaked and ground kernels; and
subjecting the germ to enzymatic treatment with at least one xylanase selected from the group consisting of; a GH5 xylanase, a GH10 xylanase, a GH30 xylanase, and a GH11 xylanases.

4. The method according to claim 3, wherein said germ is admixed with said one or more xylanase enzymes during or after step b), during step c), or after the germs have been separated.

5. The method according to claim 3, wherein said germ is admixed with said one or more xylanase enzymes after step c).

6. The method according to claim 3, wherein said germ is allowed to react with said one or more xylanase enzymes for at least 45 min, at least 1 hour, at least 2 hours at least 2.5 hours, or at least 3 hours.

7. The method according to claim 3, wherein said hydrolytic enzyme is present in an amount of 0.0005 to 1.5 mg enzyme protein per g DS kernels, 0.001 to 1 mg enzyme protein per g DS kernels, 0.002 to 0.5 mg enzyme protein per g DS kernels, or 0.003 to 0.4 mg enzyme protein per g DS kernels.

8. The method according to claim 3 wherein the hydrolytic enzymes comprises at least one xylanase and at least one arabinofuranosidase.

9. The method according to claim 3, wherein the hydrolytic enzymes comprises at least one beta-glucanase.

10. The method according to claim 3, wherein the hydrolytic enzymes further comprise cellulases.

11. The method according to claim 9, wherein the cellulases are selected from the group consisting of an endoglucanase, a cellobiohydrolase I, a cellobiohydrolases II, a GH61, a beta-glucosidase, and combinations thereof.

12. The method f according to claim 11, wherein the cellulases comprise at least an endoglucanase and a cellobiohydrolase.

13. The method according to claim 12, wherein the cellulases comprise at least a cellobiohydrolase I, a cellobiohydrolase II, and a endoglucanase I.

14. The method according to claim 13, wherein the cellulases are derived from Trichoderma.

15. (canceled)

16. The method according to claim 3, wherein the xylanase is a GH10 xylanase.

17. The method according to claim 3, wherein the xylanase is a GH5_21 xylanase.

18. The method according to claim 3, wherein the xylanase is a GH11 xylanase.

19. The method according to claim 3, wherein the xylanase is a GH30_8 xylanase.

20. The method according to claim 8, wherein the arabinofuranosidase is a GH62 or a GH43 arabinofuranosidase.

21. The method according to claim 9, wherein the beta-glucanase is a GH5_15 beta-glucanase.

22. The method according to claim 3, wherein the germ is dewatered/dried.

23. The method according to claim 3 further comprising the step of extracting oil from the germ.


SEQUENCE LISTING (7 sequences)

SEQ ID NO: 1 - protein, 405 aa, Talaromyces leycettanus
MVHLSSLALALAAGSQLAQAAGLNTAAKAIGKLYFGTATDNPELSDSTYMQETDNTDDFGQLTPANSMKWDATEPSQNTFTFTNGDQIANLAKSNGQMLRCHNLVWYNQLPSWVTSGSWTNATLLAAMKNHITNVVTHYKGQCYAWDVVNEALNDDGTYRSNVFYQYIGEAYIPIAFATAAAADPNAKLYYNDYNIEYPGAKATAAQNIVKMVKAYGAKIDGVGLQSHFIVGSTPSQSSQQSNMAAFTALGVEVAITELDIRMTLPSTSALLAQQSTDYQSTVSACVNTPKCIGITLWDWTDKYSWVPNTFSGQGDACPWDSNYQKKPAYYGILTALGGSASTSTTTTLVTSTRTSTTTSTSATSTSTGVAQHWGQCGGIGWTGPTTCASPYTCQELNPYYYQCL

SEQ ID NO: 2 - protein, 325 aa, Talaromyces pinophilus
MHFLAALLAVLPLVSGSPVPEKRSGCALPSTYKWTSTGPLASPKSGLVALRDYSHVIYNGQHLVYGSTANTAGSYGSMNFGLFSDWSEMSSASQNTMSTGAVAPTIFYFAPKSVWILAYQWGPYAFSYRTSTDPSNANGWSSPQPLFTGTISGSSTGVIDQTVIGDSENMYLFFAGDNGHIYRASMPIGDFPGSFGSASTIVLSDSTNNLFEAVEVYTVEGQNQYLMIVEAIGANGRYFRSFTASSLGGTWTAQASTESNPFAGKANSGATWTNDISSGDLVRTNPDQTQTIDACNLQFLYQGRSTSSGGDYNLLPYQPGLLTLA

SEQ ID NO: 3 - protein, 177 aa, Thermoascus aurantiacus
TRISSCSGSRQSALTTALRNAASLANAAADAAQSGSASKFSEYFKTTSSSTRQTVAARLRAVAREASSSSSGATTYYCDDPYGYCSSNVLAYTLPSYNIIANCDIFYTYLPALTSTCHAQDQATTALHEFTHAPGVYSPGTDDLAYGYQAAMGLSSSQAVMNADTYALYANAIYLGC

SEQ ID NO: 4 - protein, 551 aa, Chryseobacterium sp
MDEKNLLEDPDSNLSAGASARALAATPMLHVGGRYLKDPCDNNVLLHGVAITPSPWFNGCQYGANSGYCTWDNYNVQGCLNYNKAVMNKLTSAADGWYLNYIRLHIDPYWTNDPGPSIPENDISRFNYNRLVTYTDQVIIPLINHARSRGMYVILRPPGVCPNRIAVNDAYHSYLKTVWTFLSQHPGLKNADNVMFELANEPVEILGTNGTWGQTGNEHFAALKNFFQPLVNIIRNNGANNVCWIPGTGWQSHYQGYPNNQITGGNIGYAVHIYPSYWGGLSNYQAFQNAWNINVKPIADIAPIAITETDWAPQGYGTFGIGTTGTAGGSGFGANLKYIVDQSGNVSWNVLAPDNLLHKGDPNGGTAYNNDWEACAAPVKQWFQQYAKSNYPVGNCNTTSSLVNNGIYEIEFQTDANKVVDLKSGEDANGAVLRPWTRNGAAAQRWVAIDAGNGYWRFVSKASATNRCITLASNSNTLGTSIVLWQNYGNDAQAWQVVAVSNGYYKILSKVDPTRGWDIPNCTMDGNSNLHLWDYYGTSCQLFKFKYIGMN

SEQ ID NO: 5 - protein, 417 aa, Bacillus sp-18423
MKKPLGKIVASTALLISVAFSSSIASAASDVTVNVSAEKQVIRGFGGMNWPAWAGDLTAAQRETAFGNGQNQLGFSILRIHVDENRNNWYKEVETAKSALKLGAIVFASPWNPPSDMVETFNRNGDTSAKRLKYNKYAAYAQHLNDFVTFMKNNGVNLYAISVQNEPDYAHEWTWWTPQEMLRFMRENAGSINARVIAPESFQYLKNLSDPILNDPQALANMDILGTHLYGTQLSQFPYPLFKQKGAGKDLWMTEVYYPNSDTNSADRWPEALDVSQHIHNAMVEGDFQAYVWWYIRRSYGPMKEDGTISKRGYNMAHFSKFVRPGYVRIDATKNPNANVYVSAYKGDNKVVIVAINKSNTGVNQNFVLQNGSASNVSRWITSSSSNLQPGTNLTVSGNHFWAHLPAQSVTTFVVNR

SEQ ID NO: 6 - protein, 212 aa, Geobacillus stearothemophilus
MKQQKRLYARLVLMCTLLFVSLPITKTSAATDYWQYWTDGGGMVNAVNGPGGNYSVTWQNTGNFVVGKGWTVGSPNRVINYNAGIWEPSGNGYLTLYGWTRNALIEYYVVDSWGTYRPTGNYKGTVNSDGGTYDIYTTMRYNAPSIDGTQTFQQFWSVRQSKRPTGSNVSITFSNHVNAWRSKGMNLGSSWAYQVLATEGYQSSGRSNVTVW

SEQ ID NO: 7 - protein, 408 aa, Trichoderma atroviride
MLSAYAFSLLVASASAWTPQSNAKVFSRDTEGVMRWLPGNDKFRGVNLGSQFIIEPWMASDEFSGMGCGGLNDEWSCVQSLGQDAADAAFQKHWDSWITQDDITQIKNLGLNTVRIPVGFWIREDLVQQGEFFPRGGIQYLDRLVGWCNDAGIYVIMDLHGGPGAQFPNQQYTGHGVSQPGFYTEANYERAANFLEWMTERIHTNATYASVGMLEVINEPVHSGDFPSQAADMVNTYYPLAWNRIRDTESKLGVSDDKRLHIQFMASAWGSGDPTSALPSTDFAAFDDHRYLKWDTSVTATKDGYLNAACSDKRDDNVIVGEWSISVADNVQDNDELGIKNRSDQADWYQKFWAAQVLAFEKSAGWVFWTWKCNWITGYDDWRWCYQSAVAAGAIPKDAGSAASINPC